(19) United States
(12) Plant Patent Application Publication
(10) Pub. No.: US 2004/0133955 P1
(43) Pub. Date: Jul. 8, 2004

(54) Magnolia plant named 'JURmag2'

(76) Inventor: Mark C. Jury, North Taranaki (NZ)

Correspondence Address: MICHAEL DOLL, FULWIDER, PATTON, LEE & UTECHT, 200 OCEANGATE, SUITE 1550, LONG BEACH, CA, 90802, US

(21) Appl. No.: 10/335,927

(22) Filed: Jan. 3, 2003

(51) Int. Cl. A01H 5/00

(52) U.S. Cl. PLT/223

(57) ABSTRACT

A new and distinct cultivar of Magnolia named ‘Felix’ comprised of strong plant growth, upright and open; flowering as a young plant within 2 years of planting out, a long heavy flowering season; strongly fragrant individual flowers lasting about 9 days; ovate and large buds; external surface is typically pubescent and dark grey in colour, opening in a typical manner of all deciduous Magnolias; leaves which are a lighter green and slightly paler underneath than most Magnolia varieties and are smooth on top and lightly pubescent underneath; and the tree can set heavily with very large pods and many seeds.

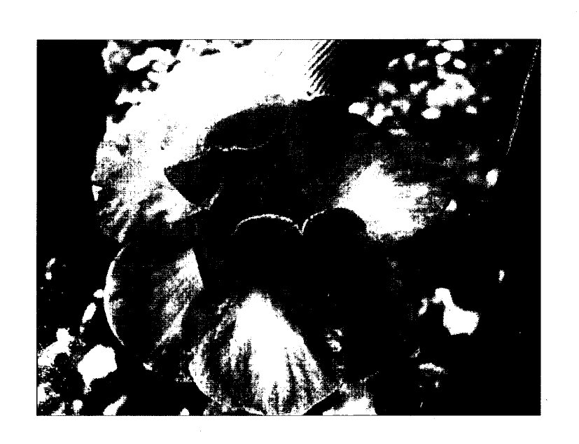

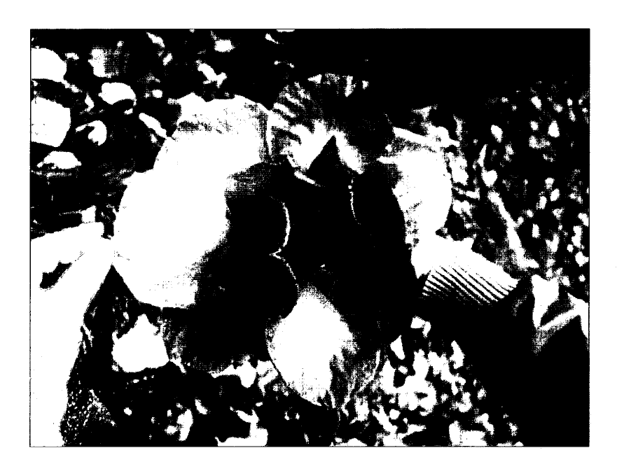

Latin Name of the Genus and Species of the Plant Claimed:

Magnolia Soulangeana

Variety Denomination:

Felix

### BACKGROUND OF THE INVENTION

The present invention relates to a new and distinct cultivar of Magnolia Soulangeana referred to by the variety denomination ‘Felix’.

The new cultivar was isolated by the inventor Mark C. Jury at North Taranaki in New Zealand, by seedling selection following controlled pollination of the maternal parent Magnolia ‘Atlas’ (unpatented) with the paternal parent Magnolia ‘Vulcan’ (unpatented). The cross was made in 1986. The new cultivar was selected from the progeny of the stated cross by the inventor with the selection being primarily on the basis of flower color and form.

Following selection of the seedling, the cultivar was grown from cuttings propagated vegetatively by grafting. Following several generations of propagated cuttings, uniformity and stability of the selected characteristics was observed. Horticultural examination of the present cultivar has demonstrated that the combination of characteristics herein disclosed for Magnolia ‘Felix’ are firmly fixed and are retained through successive generations of asexual reproduction.

The following table of characteristics indicates the manner in which the new cultivar is distinct from both Magnolia ‘Atlas’ and Magnolia ‘Vulcan’.

TABLE 1

| | | | Paternal |
|---|---|---|---|
| | | | Parent |
| | | Maternal Parent | Magnolia |
| | Magnolia Felix | Magnolia ‘Atlas’ | ‘Vulcan’ |
| Flower Color | Rosy Pink to Dark | Pale lilac and | Wine Red |
| | Rosy Red | Cream | |
| Flower Shape | Goblet Shape | Cup Shape | |
| Flower Size | 30 cm | 37.5 cm | |
| Flower Head size | to 30 cm diameter | | to 20 cm |
| | | | diameter |
| Flower Petal size | Large | | Medium |
| Plant Size | Stout Tree 7 to | Taller Tree to | |
| | 8 m | 12 m | |
| Tree Shape | Stout pyramidical | Rounded | |
| | shape | | |
| Growth Habit | Finer Growth | Single Trunk | More Open |
| | Habit, More | | |
| | dense | | |

Magnolia ‘Vulcan’ is the closest comparison variety known to the applicant, but is quite different as noted in Table 1 above.

### BRIEF SUMMARY OF THE INVENTION

The following traits have been repeatedly observed and are determined to be basic characteristics of Magnolia ‘Felix’, which in combination distinguish this Magnolia Soulangeana as a new and distinct cultivar:

1. Plant growth is strong, upright and open;

2. Flowers as a young plant within 2 years of planting out, with a long heavy flowering season;

3. Individual flowers are strongly fragrant, and last about 9 days;

4. Buds are ovate and large; external surface is typically pubescent and dark grey in colour, opening in a typical manner of all deciduous Magnolias;

5. Leaf of the plant is a lighter green and slightly paler underneath than most Magnolia varieties and is smooth on top and lightly pubescent underneath; and

6. The tree can set heavily with very large pods and many seeds.

The following observations, measurements, and values describe the new cultivar as grown in Silvan, Victoria, Australia under conditions which approximate those generally used in commercial practice.

### BRIEF DESCRIPTION OF THE DRAWINGS

The accompanying color photographic drawings show the unique characteristics of the new cultivar Magnolia ‘Felix’, with colors as accurate as is possible with color photographs. The first photographic drawing shows a top view of an individual inflorescence of ‘Felix’. The second photographic drawing shows a top view of an individual inflorescence of ‘Felix’, emphasizing the petal.

### DETAILED BOTANICAL DESCRIPTION

Color references are made to The Royal Horticultural Society Colour Chart (RHS) except where general colors of ordinary significance are used. Colors are approximate as color depends on horticultural practices such as light level and fertilization rate among other things.

### ORIGIN

Seedling from breeding program.

### PARENTAGE

Maternal parent — Magnolia hybrid ‘Atlas’.

Paternal parent — Magnolia hybrid ‘Vulcan’.

### PROPAGATION

Plants have been selected from the original seedling and propagated vegetatively by grafting in New Zealand

### PLANT

Growth rate: Strong growth rate. Flowers as young plant within 2 years of planting out.

Length of flowering season: Long.

Time of flowering season: Medium.

Density: Open.

Growth type: Pyramidial.

Height: 7 m.

Width: 4 m.

Stem diameter: 250 mm.

### LEAF

Color: RHS Yellow Green Group — 146B; underside of leaf is sightly paler.

Length: 250 mm.

Width: 200 mm.

Apex: Acuminate.

Margin: Entire.

Texture: Smooth on top, lightly pubescent underneath.

Shape: Ovate.

Petiole: 30 mm in length; RHS Yellow/Green Group — 146C.

### STEM

Branching arrangement: Open.

Surface texture: Smooth.

Lenticels: 2-3 mm (small); RHS Yellow Group — 11D.

### FLOWER

Length of flowering period: Long.

Number of flowers: Many.

Floret length: 200 mm.

Lastingness of bloom: Approximately 9 days.

Fragrance of bloom: Typical fragrance of Magnolia but stronger.

Flower profile upper: Flat.

Flower profile lower: Convex.

Color midzone outside: RHS Rose Red.

Midzone inside: RHS Rose Red.

Margin inside: RHS Rose Red.

Buds:

Bud shape.—Ovate.

Petals:

Number of petals.—12.

Petal size.—Large.

Petal width.—150 mm.

Petal arrangement.—Overlapping.

Pistils:

Style color.—Rose Red.

Seed vessel characteristics:

Size.—Large.

Color.—Red.

1. A new and distinct magnolia plant named ‘Felix’, as illustrated and described herein.
